(19) United States
(12) Patent Application Publication
(10) Pub. No.: US 2012/0088965 A1
(43) Pub. Date: Apr. 12, 2012

(54) MAGNETICALLY MANIPULATABLE SURGICAL CAMERA WITH REMOVABLE ADHESION REMOVAL SYSTEM

(75) Inventors: Michael J. Stokes, Cincinnati, OH (US); William B. Weisenburgh, II, Maineville, OH (US); Alexander P. Kondor, Cincinnati, OH (US); Christopher J. Hess, Cincinnati, OH (US)

(73) Assignee: Ethicon Endo-Surgery, Inc., Cincinnati, OH (US)

(21) Appl. No.: 12/902,531

(22) Filed: Oct. 12, 2010

(51) Int. Cl. A61B 1/00

(52) U.S. Cl. 600/104

(57) ABSTRACT

A surgical or diagnostic camera system is disclosed that has a disposable cap-like device that is structured for removal of adhesions while under direct visualization with the camera. The adhesion removal device may have an electrode on the tip that can be activated to enhance separation of tissues and may be configured to securely snap on to the housing of the camera system.

### BACKGROUND

i. Field of the Invention

The present application relates to methods and devices for use in minimally invasive diagnostic, therapeutic and surgical procedures and, more particularly, to a device for use with a surgical camera to remove adhesions.

ii. Description of the Related Art

An adhesion is a band of scar tissue that binds two parts of tissue together that naturally should be separate. Adhesions may appear as thin sheets of tissue similar to plastic wrap or as thick fibrous bands. The tissue develops when the body's repair mechanisms respond to any tissue disturbance, such as surgery, infection, trauma, or radiation. Although adhesions can occur anywhere, the most common locations are within the stomach, the pelvis, and the heart.

Abdominal adhesions are a common complication of surgery, occurring in up to 93% of people who undergo abdominal or pelvic surgery. Abdominal adhesions also occur in over 10% of people who have never had surgery. Most adhesions are painless and do not cause complications. However, adhesions cause 60%-70% of small bowel obstructions in adults and are believed to contribute to the development of chronic pelvic pain. Adhesions typically begin to form within the first few days after surgery, but they may not produce symptoms for months or even years. As scar tissue begins to restrict motion of the small intestines, passing food through the digestive system becomes progressively more difficult. The bowel may become blocked. In extreme cases, adhesions may form fibrous bands around a segment of an intestine. This constricts blood flow and leads to tissue death.

The benefits of minimally invasive procedures compared to open surgery procedures for treating certain types of wounds and diseases are now well-known to include faster recovery time and less pain for the patient, better outcomes, and lower overall costs. In minimally invasive surgical procedures, such as laparoscopic surgery, a surgeon may place one or more small ports into a patient's abdomen to gain access into the abdominal cavity of the patient. A surgeon may use, for example, a port for introducing a laparoscope for viewing, and a number of other ports for introducing surgical instruments for operating on tissue. Other minimally invasive surgical procedures include natural orifice transluminal endoscopic surgery (NOTES) wherein surgical instruments and viewing devices are introduced into a patient's body through, for example, the mouth, nose, or rectum.

New viewing systems for minimally invasive techniques have been developed. In all of these viewing systems, however, the interference of adhesions remains a problem for many patients.

The foregoing discussion is intended only to illustrate various aspects of the related art in the field of the invention at the time, and should not be taken as a disavowal of claim scope.

### SUMMARY

An effective means of removing adhesions under direct view has been developed. An adhesion removal device is described herein for use with an instrument, such as a camera. The adhesion removal device has a tip portion on a leading end of the device. When mounted on the housing of an instrument, the tip portion extends outwardly from the first end of the housing. The tip portion is structured for prying the bound tissue of an adhesion apart.

The adhesion removal device may have a first end and a second end wherein the tip portion is positioned on the first end of the removal device. An electrode may also be positioned on the first end of the removal device. An energy tether may extend from the second end of the removal device for operative connection to a source of energy. The removal device may also have a channel along the perimeter thereof for carrying a wire for the transfer of energy from the energy tether to the electrode.

In one embodiment, the removal device has engagement surfaces for releasable attachment to the housing of an instrument. The engagement surfaces may comprise a pair of prongs for grasping the housing. Alternatively, the housing may have engagement surfaces configured for releasable complementary engagement with the engagement surfaces of the removal device.

The removal device may be made of a biocompatible material, and in certain embodiments, may be made of a transparent biocompatible nonmagnetic material.

An adhesion removal system may include a housing having a first end and at least one camera positioned on the first end, and the adhesion removal device releasably mounted on the housing. The housing may have a body portion adjacent the first end.

For purposes of orientation, the housing may be described as having a central, longitudinal axis and in use, the housing would be positioned within the patient such that the longitudinal axis would be generally parallel and preferably substantially parallel to the abdominal wall of the patient undergoing the procedure. The longitudinal axis of the housing defines the zero degree angle. A plane passing between the first end of the housing and the adjacent body portion perpendicular to the longitudinal axis of the housing and generally perpendicular and preferably substantially perpendicular to the abdominal wall when in use, would define a ninety degree angle. For purposes of orientation, the point of intersection between the perpendicular plane and the central longitudinal axis of the housing is the origin.

In certain embodiments, the housing carries one or more cameras and may carry other instruments, lighting and circuit boards with controls for the camera and lights, as desired. One camera has a lens that may be directed at an angle greater than zero degrees and less than 90 degrees, preferably between about 10 and 60 degrees, more preferably between 10 and 45 degrees, still more preferably between 25 and 35 degrees, and most preferably at or about an angle of 30 degrees downwardly relative to the origin and the longitudinal and perpendicular axes.

In another embodiment of the adhesion removal system, there may be two cameras on the housing and the first of the cameras may have a lens directed at an angle of between about 10 and 60 degrees, more preferably between 10 and 45 degrees, still more preferably between 25 and 35 degrees, and most preferably at or about an angle of 30 relative to the origin and the longitudinal and perpendicular axes, and the second of the cameras may have a lens directed at an angle of between 0 and 90 degrees, different from the angle of the first camera lens. For example, one camera lens may be directed along one of the central axis of the housing or an axis parallel to the central axis of the housing, at 0 degrees, and the other camera may have a lens directed at an angle of 30 degrees relative to the central axis of the camera from the point of intersection.

The housing may include at least one light source adjacent to the at least one camera.

The housing may be part of a magnetic guidance system and may include magnets mounted therein. The adhesion removal system of the magnetic guidance system may further include an external magnetic control unit for manipulating the movement of the housing and the camera when deployed in use in a patient.

The adhesion removal device may be removably attached to the housing by such means as adhesive material, snap on feature, a dove tail feature, magnetically coupled, or other well known attachment means.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 shows a side elevation view of an embodiment of an adhesion removal device.

FIG. 2 shows a side elevation view of a housing carrying two cameras.

FIG. 3 shows a side elevation view of the adhesion removal device of FIG. 1 attached to the housing of FIG. 2.

FIG. 4 shows a perspective view of the combination of an adhesion removal device and an alternative embodiment of the housing having one camera.

FIG. 5 shows the adhesion removal device and housing combination of FIG. 3 in use removing an adhesion at a site within a patient during a surgical or diagnostic procedure.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

As used herein, the term “biocompatible” includes any material that is compatible with the living tissues and system(s) of a patient by not being substantially toxic or injurious and not causing immunological rejection. “Biocompatibility” includes the tendency of a material to be biocompatible.

As used herein, the term “longitudinal axis”, with respect to an instrument, means the exact or approximate central axis defined by said instrument along its greater dimension, i.e., along its length, from its distal end to its proximal end, and vice versa, and is not intended to be limited to imply a straight line, wherein, for example, an instrument includes a bend angle as described herein, it is intended that “longitudinal axis” as used herein follows such bend angle. As used herein, the term “axial” or “axial movement” or variants thereof, with respect to an instrument or a component of an instrument, means the movement in the direction of the longitudinal axis of such instrument.

As used herein, the term “patient,” used herein, refers to any human or animal on which a suturing procedure may be performed. As used herein, the term “internal site” of a patient means a lumen, body cavity or other location in a patient's body including, without limitation, sites accessible through natural orifices or through incisions.

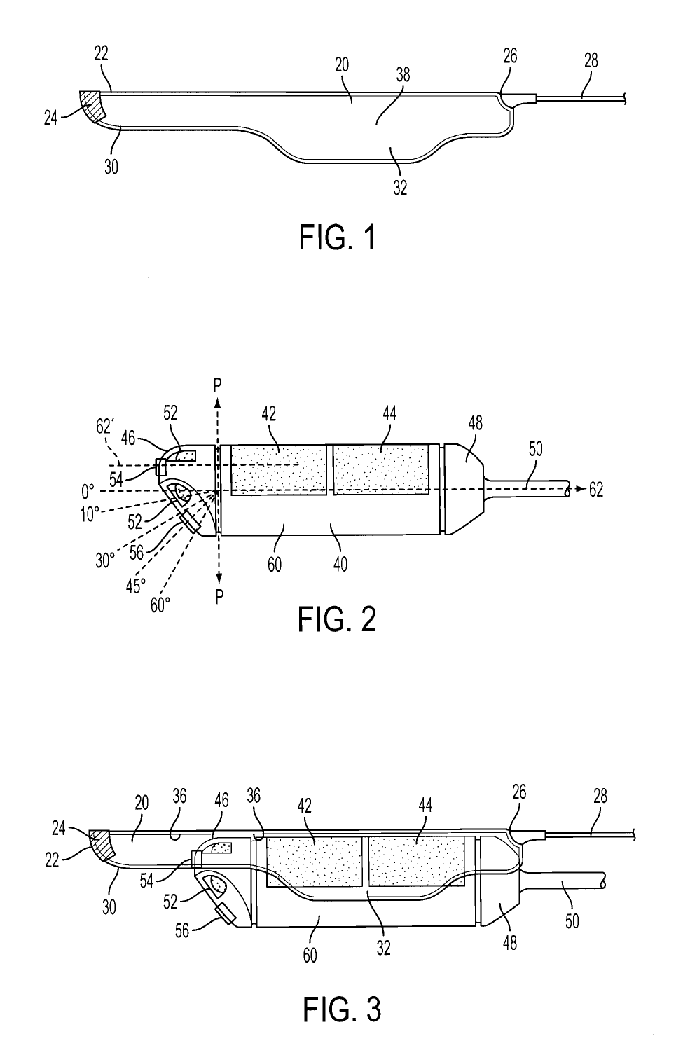

FIG. 1 is a view of an embodiment of an adhesion removal device 20. In the embodiment shown, the device 20 includes a body portion 38 having a pair of opposing prongs 32, a leading tip 22 and a trailing end 26. Tip 22 may be shaped with a beveled or thin edge to tease or pry apart the tissue of an adhesion to separate the two bound areas of tissue. The opposing prongs 32 (only one side is shown) are structured to attach to a housing body, such as the housing used in a magnetic anchoring and guidance system (MAGS) that is shown in FIG. 2.

The adhesion removal device of FIG. 1 may also include an electrode 24 on the leading tip 22 connected by wires 30 that run along a channel on the perimeter of the body portion 38 to an energy based tether 28, such as an insulated electrical wire, that extends from the trailing end 26 of the device 20 for connection with an energy source (not shown). In use, when the adhesion removal device 20 is deployed in a patient during a minimally invasive surgical or diagnostic procedure, the tether 28 would typically pass through a port in a trocar, endoscope, laparoscope, or other port (not shown) from the inside to the outside of a patient's body directly, or indirectly through an intermediate instrument, to an energy source. Alternatively, the tether could pass along the outside of a port, between the incision and trocar. If the adhesion will not come apart by mechanical measures alone using the tip 22, the electrode 24 may be energized to rapidly heat the tissue of the adhesion to complete the adhesion removal. Those skilled in the art will appreciate that when hot tips (monopolar electrocautery), such as electrode 24, are used in surgical procedures, a ground pad is placed under the patient. By applying electricity to the electrode 24, resistance is created between the electrode and the ground pad, resulting in the rapid heating of the surrounding tissue but not heating the electrode itself. The tether 28 may be operatively connected to a control device, controllable by means of a foot pedal, hand control or the like by the clinician or other operating room personnel.

An embodiment of the MACS instrument housing with a camera mounted thereon for minimally invasive surgical or diagnostic procedures is shown in FIG. 2. The camera can be used to observe surface conditions of internal organs, including abnormal or diseased tissue such as lesions and other surface conditions, and can capture images for visual inspection and photography for taking biopsies, retrieving foreign objects, and/or performing surgical or diagnostic procedures.

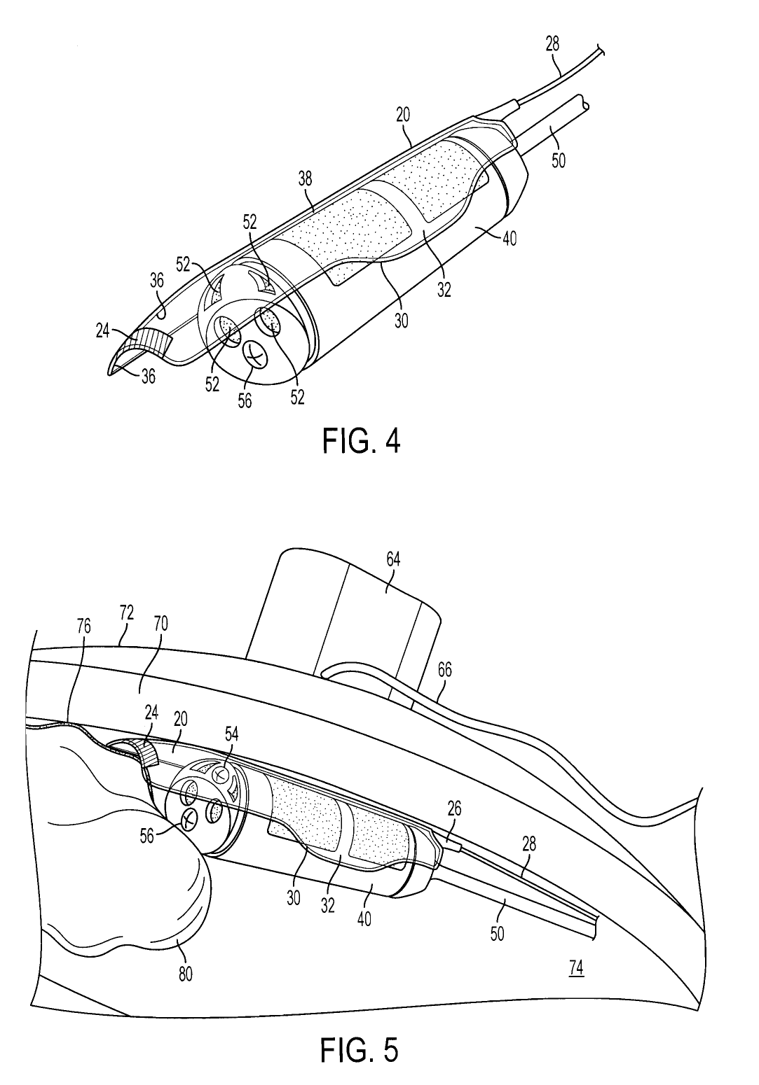

Referring to FIG. 2, the housing 40 includes a central longitudinal axis 62 through the length of the housing, a body portion 60, shown as generally tubular in shape, a leading head portion 46 and a trailing end portion 48. Housing 40 may include at least one camera and at least one light emitting diode (LED). In the embodiment of housing 40 shown in FIG. 2, there are two LEDs 52 for each of the two cameras 54 and 56 on head portion 46. In the embodiment shown in FIG. 4, there is one camera 56.

For purposes of orientation, there is a plane P perpendicular to the longitudinal axis 62, between the body portion 60 and the head portion 46 of the housing 40. The intersection between plane P and the axis 62 shall be referred to herein as the origin, O. For purposes of orientation, the orientation of the lens of the cameras are described herein as directed at angles relative to the axis 62, plane P and origin, O.

The housing 40 may include a camera 56 having a lens directed at an angle greater than 0° and less than 90° and preferably between 10° to 60°, more preferably between 10° to 45°, measured downwardly, or distally, from the longitudinal axis 62 for viewing tissue under the axis 62 of the housing 40. In one embodiment, the angle of the camera 56 lens relative to the central axis 62 is directed between 20° and 40°, and more preferably between about 25° and 35°, and most preferably at or about 30°. The housing 40 may have in addition, a camera 54 having a lens aligned with the axis 62 or with a line parallel to it, at or about 0 degrees along the axis 62 for viewing sites directly ahead of the housing 40. Those skilled in the art will appreciate that the cameras 54, 56 as used in the housing 40 may be any known optical viewing systems, such as, without limitation, standard cameras and lights, or fiber optic systems, or CCD systems.

A tether 50 extends from the trailing end 48 of the housing 40. Like tether 28 of the adhesion removal device 20, the tether 50 may be an energy tether, such as an insulated electrical wire that extends from the trailing end 48 of the housing 40 for connection with an energy source (not shown). Tether 50 may also carry video images to a video screen outside of the patient. In use, when the housing 40 is deployed in a patient during a minimally invasive surgical or diagnostic procedure, the tether 50 would typically pass through a port (not shown) from the inside to the outside of a patient's body directly, or indirectly through an intermediate instrument, to an energy source or a receiver or processor for receiving video signals from the one or more cameras.

The adhesion removal system comprising the combined housing 40 with the adhesion removal device 20 attached is shown in FIGS. 3 and 4. The body 38 of device 20 may be shaped to conform generally to the shape of at least the top portion 58 of the housing body 60 with the open area between opposing prongs 32 decreasing in width, or curvature, so that prongs 32 allow device 20 to snap onto, or otherwise grasp, the sides of housing body 60 when attached. The underside 36 of adhesion removal device 20 may conform to the shape of at least top portion 58, and most of body 60 of housing 40. Alternatively, the housing 40 may have an engagement surface on the top portion 58 or along each side configured to mate with a complementary engagement surface on, for example, each prong 32 or on the underside 36 of the removal device 20 to mechanically secure the device 20 onto housing 40 during use within a patient's body. The engagement surfaces may be any well known complementary engagement system, such as a rail and channel arrangement, or dovetails, hooks, snaps and the like. The adhesion removal devise 20 may be attached to housing 40 magnetically or with an adhesive.

The leading tip 22 of device 20 narrows to avoid blocking the view of camera 54 or the light emitted from the LEDs 52. Tip 22 is preferably positioned above and to the periphery of the line of sight of the camera 54 to avoid blocking the view of the lens of camera 54 with the edge of tip 22 or the electrode 24. The central axis of tip 22 may therefore be parallel to axis 62 of housing 40 such that tip 22 is substantially or completely straight as it extends from the body 38.

The adhesion removal device 20 may be made of a clear plastic material that allows light or signals to pass through substantially unimpeded. Alternatively, device 20 may be opaque or dark to prevent the passage of light. When the housing 40 carries a MACS camera, the device 20 must be made of a material that does not interfere with the magnetic attraction between the internal magnets 42, 44 on housing 40 and the external magnets on external control device 64. (see FIG. 5). Exemplary materials include biocompatible plastic materials, such as polycarbonate, Plexiglas or nylon, or other biocompatible non magnetic materials. Suitable materials are commercially available. The leading edge of the adhesion removal device may be made of an electrically conductive material to pass energy to the adhesion.

In an alternative embodiment, the adhesion removal device 20 may not include all of body portion 38 but may instead comprise only a portion equivalent to the leading tip 22 that attaches to housing 40, or to the head portion 46 of housing 40, to position the tip 22 ahead and above the cameras 54, 56 to remove adhesions under direct view of a camera. Means for running the wire 30 from the tether 28 to the electrode 24 in those embodiments having an electrode 24 may comprise flexible, resilient channel members that both house the wires 30 and grasp the housing 40 to secure the tip 22 in place during use. Suitable engagement surfaces, such as those described above, to releasably secure device 20 to housing 40 may be used. In another alternative embodiment, the adhesion removal tip 22, and optionally the electrode 24, may be fixed to the head portion 46 of housing 40.

Referring to FIG. 5, the combination of the housing 40 and adhesion removal device 20 is shown in use in a patient's body, for example the abdomen, removing an adhesion 76 between the tissue of organ 80 and the tissue 74. The MACS camera system is not steered like the traditional handheld camera with a long rigid shaft attached to a camera processor. The MACS camera system is deployed in the body and then picked up by a magnetic, external control unit 64. The external control unit 64 is on the exterior side 72 of the abdominal wall 70 and is guided around by the surgeon or clinician until the camera is positioned in the critical surgical site. Having an adhesion could prevent the surgeon from being able to properly position the camera to view the desired site.

The housing 40 shown in FIG. 5 is a MACS camera that is manipulated by movement of the external control unit 64 on the exterior 72 of the patient. External control unit 64 includes large permanent magnets (not shown) that magnetically attract the magnets 42, 44 on the housing 40. External control unit 64 may be powered through electronic tether 66 which may be attached, directly or indirectly, to a power source.

As shown in FIG. 5, when there is an adhesion 76, the device 20, which is releasably attached to housing 40 by prongs 32 is advanced towards the adhesion 76 when movement of external control device 64 moves housing 40. The images of the adhesion 76 and surrounding tissue 74 are viewed in real time by the clinician who controls the movement of external control device 64 based at least in part on the images communicated, in this embodiment, outside of the patient via the tether 50 trailing housing 40 to a viewing screen or monitor. If application of mechanical pressure against the adhesion by pushing the edges of tip 22 against the adhesion is not sufficient to remove the adhesion, the clinician may activate the energy supply to electrode 24 by any suitable means, such as depressing a foot pedal control or an activation switch on a hand held device or another control device. The energy supplied to the electrode 24 will generate sufficient heat in the adhesion to separate the bound tissue.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, nose, anus, and/or vagina, for example or via a trocar through a relatively small—keyhole—incision incisions (usually 0.5-2.5 cm). Minimally invasive procedures performed by the introduction of various medical devices into the patient through a natural opening of the patient are known in the art as NOTES™ procedures.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam. The housing 40 is intended for re-use so will always have to be sterilized before use and thoroughly cleaned after each use. The adhesion removal device 20 is preferable a disposable component that would be sterile before use and disposed of by acceptable biohazard disposal techniques following use.

Except as otherwise noted, the articles “a”, “an”, and “the” mean “one or more”.

Except as otherwise noted, all amounts including quantities, percentages, portions, and proportions, are understood to be modified by the word “about”, and amounts are not intended to indicate significant digits.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Although the various embodiments of the devices have been described herein in connection with certain disclosed embodiments, many modifications and variations to those embodiments may be implemented. For example, different types of instruments may be employed in the housing. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An adhesion removal device comprising:
an engagement surface for releasable attachment to a housing; and
a tip portion structured when attached to the housing to extend outwardly from a leading end of the housing, the tip portion having an edge for prying bound tissue of an adhesion apart under direct view of a camera.

2. The adhesion removal device recited in claim 1 having a first end and a second end, the tip portion being positioned on the first end of the removal device, and further comprising an electrode on the first end and an energy tether extending from the second end for operative connection to a source of energy.

3. The adhesion removal device of claim 2 having a channel along the perimeter thereof for carrying a wire for the transfer of energy from the energy tether to the electrode.

4. The adhesion removal device of claim 1 having engagement surfaces for releasable attachment to the camera.

5. The adhesion removal device of claim 4 wherein the engagement surfaces of the removal device comprise a pair of prongs for grasping the housing.

6. An adhesion removal system comprising:
a housing having a first end and at least one camera positioned on the first end; and
an adhesion removal device releasably mounted on the housing and having a tip portion that extends outwardly from the first end of the housing structured for prying bound tissue of an adhesion apart.

7. The adhesion removal system of claim 6 wherein the removal device has a first end and a second end, the tip portion being positioned on the first end of the removal device, and further comprising an electrode on the first end and an energy tether extending from the second end for operative connection to a source of energy.

8. The adhesion removal system of claim 7 wherein the removal device has a channel along the perimeter thereof for carrying a wire for the transfer of energy from the energy tether to the electrode.

9. The adhesion removal system of claim 6 wherein the removal device has engagement surfaces for releasable attachment to the housing.

10. The adhesion removal system of claim 9 wherein the engagement surfaces of the removal device comprise a pair of prongs for grasping the housing.

11. The adhesion removal system of claim 9 wherein the housing has engagement surfaces configured for releasable complementary engagement with the engagement surfaces of the removal device.

12. The adhesion removal system of claim 6 wherein the removal device is made of a transparent biocompatible nonmagnetic material.

13. The adhesion removal system of claim 6 wherein the removal device is made of a biocompatible material.

14. The adhesion removal system of claim 6 wherein the housing has one camera having a lens directed at an angle greater than 0 degrees and less than 90 degrees downwardly from the central axis of the camera relative to such axis.

15. The adhesion removal system of claim 14 wherein the housing has a second camera.

16. The adhesion removal system of claim 15 wherein the second camera has a lens directed along one of the central axis of the housing or an axis parallel to the central axis of the housing.

17. The adhesion removal system of claim 14 wherein the camera lens is directed at an angle between 10 and 45 degrees downwardly from the central axis relative to the central axis of the housing.

18. The adhesion removal system of claim 6 wherein the housing further comprises at least one light source adjacent to the at least one camera.

19. The adhesion removal system of claim 6 wherein the housing further comprises magnets mounted therein.

20. The adhesion removal system of claim 19 further comprising an external magnetic control unit for manipulating the movement of movement of the housing when deployed in use in a patient.
